(19) United States
(12) Patent Application Publication
(10) Pub. No.: US 2021/0391077 A1
(43) Pub. Date: Dec. 16, 2021

(54) MEDICAL INFORMATION PROCESSING SYSTEM, MEDICAL INFORMATION PROCESSING APPARATUS, AND MEDICAL INFORMATION PROCESSING METHOD

(71) Applicant: Sony Group Corporation, Tokyo (JP)

(72) Inventors: Takayoshi HIRAI, Tokyo (JP); Yukako FUJIMOTO, Tokyo (JP); Kento NAKADA, Tokyo (JP)

(73) Assignee: Sony Group Corporation, Tokyo (JP)

(21) Appl. No.: 17/283,569

(22) PCT Filed: Oct. 18, 2019
(86) PCT No.: PCT/JP2019/041006

§ 371 Date: Apr. 8, 2021

(30) Foreign Application Priority Data
Oct. 19, 2018 (JP) 2018-197717

(51) Int. Cl. G16H 50/20; G16H 10/60; G16H 40/20; G16H 15/00; G06F 21/62

(52) U.S. Cl. CPC G16H 50/20; G16H 10/60; G06F 21/6254; G16H 15/00; G16H 40/20

(57) ABSTRACT

To make it possible to more suitably suppress increasing medical expenses. There is provided a medical information processing system including: an acquisition section (110 and 122); and a calculation section (122). The acquisition section (110 and 122) acquires examination information and system information. The examination information pertains to an examination from which an examination deliverable regarding medical care is generated. The system information pertains to each of a plurality of estimation systems. The plurality of estimation systems each estimates a symptom of a subject on the basis of the examination deliverable. The calculation section (122) calculates a use priority of each of the plurality of estimation systems on the basis of the examination information and the system information.

### TECHNICAL FIELD

The present disclosure relates to a medical information processing system, a medical information processing apparatus, and a medical information processing method.

### BACKGROUND ART

In recent years, increasing medical expenses have been an issue and a method of suppressing increasing medical expenses has been desired. For example, PTL1 below proposes a technique of detecting whether or not a person guided to visit a medical institution actually has a medical check or medical treatment at the medical institution and guiding the person again as necessary to prevent the person from having a severer symptom, thereby suppressing increasing medical expenses.

### CITATION LIST

### Patent Literature

PTL 1: Japanese Unexamined Patent Application Publication No. 2004-164173

### SUMMARY OF THE INVENTION

### Problems to be Solved by the Invention

The technique described in PTL1 and the like, however, request people to visit medical institutions in a large number of cases. The technique described in PTL1 and the like are insufficient as a solution to increasing medical expenses. More specifically, to estimate symptoms of subjects on the basis of a variety of examinations and examination deliverables that are results of the examinations, the subjects are requested to visit medical institutions such as hospitals in a large number of cases. The medical institutions are thus requested to have considerable resources (such as doctors and facilities, for example). This leads to increasing medical expenses. Further, for example, as second opinions are being more popular, medical expenses seem to tend to increase more.

Accordingly, the present disclosure has been devised in view of the circumstances described above. The present disclosure provides a novel and improved medical information processing system, medical information processing apparatus, and medical information processing method each of which makes it possible to more suitably suppress increasing medical expenses.

### Means for Solving the Problems

According to the present disclosure there is provided a medical information processing system including: an acquisition section; and a calculation section. The acquisition section acquires examination information and system information. The examination information pertains to an examination from which an examination deliverable regarding medical care is generated. The system information pertains to each of a plurality of estimation systems. The plurality of estimation systems each estimates a symptom of a subject on the basis of the examination deliverable. The calculation section calculates a use priority of each of the plurality of estimation systems on the basis of the examination information and the system information.

In addition, according to the present disclosure, there is provided a medical information processing apparatus including: an acquisition section; and a calculation section. The acquisition section acquires examination information and system information. The examination information includes information pertaining to an examination from which an examination deliverable of a subject regarding medical care is generated. The system information includes information pertaining to each of a plurality of estimation systems. The plurality of estimation systems each estimates a symptom of the subject on the basis of the examination deliverable. The calculation section calculates a use priority of each of the plurality of estimation systems on the basis of the examination information and the system information.

In addition, according to the present disclosure, there is provided a medical information processing method that is executed by a computer. The medical information processing method includes: acquiring examination information and system information; and calculating a use priority of each of a plurality of estimation systems on the basis of the examination information and the system information. The examination information includes information pertaining to an examination from which an examination deliverable of a subject regarding medical care is generated. The system information includes information pertaining to each of the plurality of estimation systems. The plurality of estimation systems each estimates a symptom of the subject on the basis of the examination deliverable.

### Effects of the Invention

As described above, according to the present disclosure, it is possible to more suitably suppress increasing medical expenses.

It is to be noted that the effects described above are not necessarily limitative. Any of the effects indicated in this description or other effects that may be understood from this description may be attained in addition to the effects described above or in place of the effects described above.

### BRIEF DESCRIPTION OF DRAWING

FIG. 1 is a diagram illustrating a system configuration example of a medical information processing system according to an embodiment of the present disclosure.

FIG. 2 is a block diagram illustrating a functional component example of a matching server 100.

FIG. 3 is a block diagram illustrating a functional component example of a management server 200.

FIG. 4 is a block diagram illustrating a functional component example of an estimation server 400.

FIG. 5 is a block diagram illustrating a functional component example of a user terminal 500.

FIG. 6 is a sequence diagram illustrating a flow of processes of matching an estimation system with a subject.

FIG. 7 is a sequence diagram illustrating an example of a flow of processes regarding estimation of a symptom.

FIG. 8 is a diagram illustrating an example of a user interface that is used to generate a matching request.

FIG. 9 is a diagram illustrating an example of the user interface that is used to generate the matching request.

FIG. 10 is a diagram illustrating an example of the user interface that is used to generate the matching request.

FIG. 11 is a sequence diagram illustrating a flow of processes of matching an estimation system according to a modification example with a subject.

FIG. 12 is a sequence diagram illustrating the flow of processes of matching the estimation system according to the modification example with a subject.

FIG. 13 is a block diagram illustrating a hardware configuration example of an information processing apparatus 900 that embodies the matching server 100, the management server 200, an examination terminal 300, the estimation server 400, or the user terminal 500.

### MODES FOR CARRYING OUT THE INVENTION

The following describes a preferred embodiment of the present disclosure in detail with reference to the accompanying drawings. It is to be noted that, in this description and the drawings, constitutional elements that have substantially the same functional component are indicated by the same reference signs and redundant description thereof is thus omitted.

It is to be noted that description is given in the following order.

### 1. Embodiment

### 1.1. System Configuration

### 1.2. Functional Component of Apparatus

### 1.3. Flow of Processes

### 1.4. User Interface

### 2. Modification Example

### 3. Hardware Configuration

### 4. Conclusion

### 1. Embodiment

### (1.1. System Configuration)

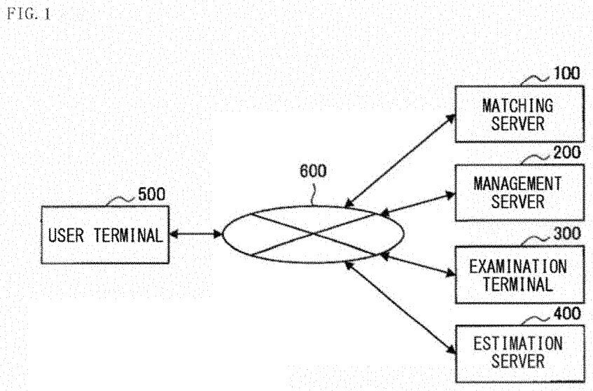

First, a system configuration example of a medical information processing system according to an embodiment of the present disclosure is described with reference to FIG. 1.

As illustrated in FIG. 1, the medical information processing system according to the present embodiment includes a matching server 100, a management server 200, an examination terminal 300, an estimation server 400, and a user terminal 500. These apparatuses are coupled by a network 600.

### (Matching Server 100)

The matching server 100 is a medical information processing apparatus that matches an estimation system with a subject. The estimation system estimates a symptom of the subject on the basis of an examination deliverable generated from an examination regarding medical care. More specifically, the matching server 100 acquires examination information regarding an examination and system information. The system information pertains to each of a plurality of estimation systems that each estimate a symptom of a subject on the basis of an examination deliverable. The matching server 100 then calculates the use priorities of the plurality of estimation systems on the basis of the examination information and the pieces of system information and outputs information (the information is information regarding a matching result and information regarding a recommended estimation system, which is sometimes referred to as “recommendation information” below) regarding at least one of the plurality of estimation systems to the user on the basis of the use priorities.

Here, the “examination regarding medical care” refers to a general action taken to evaluate the condition of a subject and the action is taken separately from the estimation of a symptom of the subject. More specifically, the examination regarding medical care includes an examination for evaluating the physical condition of a subject, an examination for determining whether or not the subject has a specific injury and illness and how severe the specific injury and illness are, and the like. The examination regarding medical care includes an examination carried out by a person in charge of medical examinations with a predetermined examination device, and the like. It is to be noted that the contents of the examination regarding medical care are not limited thereto. The following sometimes refers to the examination regarding medical care simply as “examination”.

The “examination deliverable” is information generated from an examination regarding medical care (or in the process of an examination regarding medical care). The “examination deliverable” includes, for example, captured image information (such as CT (Computed Tomography) image information or MRI (Magnetic Resonance Imaging) image information) or numerical information (such as height, weight, body fat, BMI (Body Mass Index), body temperature, visual acuity, auditory acuity, blood pressure, or blood components). It is to be noted that the contents of the examination deliverable are not limited thereto.

The “examination information” refers to some information regarding an examination regarding medical care. The “examination information” includes at least one of information (such as information regarding the type or an item of the examination, the date and time of the examination, the institution at which the examination is carried out, the medical staff member who carries the examination, or the subject who undergoes the examination, for example) regarding the details of the examination, information (such as information regarding the product name, product number, serial number, version, or manufacturer of the device used for the examination, for example) regarding a device used for the examination, or information (such as the type, data format, or data size of an examination deliverable or the number of data files of an examination deliverable, for example) regarding the details of an examination deliverable. It is to be noted that the contents of the examination information are not limited thereto.

The “estimation system” is an estimation system based on an artificial intelligence algorithm. For example, the “estimation system” is a system that is a machine learning algorithm which is one of artificial intelligence algorithms. The “estimation system” uses an examination deliverable to estimate a symptom of a subject. For example, the estimation system is a program or the like generated by performing machine learning on the basis of learning data in which an examination deliverable and a symptom are associated.

The “system information” refers to some information regarding an estimation system. The “system information” includes at least one of information (such as information regarding a device, an examination deliverable, or the like used to generate learning data of a machine learning algorithm, for example) regarding data that is used for an artificial intelligence algorithm which is used for an estimation system, information (such as an injury and illness, for example) regarding a symptom estimatable by an estimation system, information (such as a symptom or an examination deliverable for which it is possible to achieve high accuracy, for example) regarding a characteristic of an estimation system, or information (such as the type, data format, or data size of an examination deliverable, or the number of data files of an examination deliverable, for example) regarding an examination deliverable necessary for an estimation system to estimate a symptom. It is to be noted that the contents of the system information are not limited thereto.

The “use priority” is information indicating the degree to which each of estimation systems is recommended in a case where a symptom of a subject is estimated. For example, the use priority may be quantitative information such as a numerical value or qualitative information such as “high”, “medium”, and “low”. The calculation of use priorities makes it possible to provide a user with information regarding an estimation system having a higher use priority.

The matching server 100 calculates the use priorities of a plurality of estimation systems on the basis of examination information and pieces of system information and outputs recommendation information to a user on the basis of the use priorities. This allows the user to select a more suitable estimation system. In other words, a user is able to use an estimation system having higher accuracy among a plurality of estimation systems. This makes it possible to suppress the personnel expenses for a diagnostician, for example, by preventing a subject from going to a medical institution or gaining a second opinion from the estimation system, for example, in a case where the subject develops a minor symptom. It is thus possible to more suitably suppress increasing medical expenses.

In a case where a user uses the user terminal 500 to select an estimation system, the matching server 100 receives information (that is sometimes referred to as “selection information” below) indicating the selected estimation system from the user terminal 500 and transmits an examination deliverable to the estimation system (estimation server 400 that embodies an estimation system) whose use is determined on the basis of the selection information. This allows the estimation server 400 described below to estimate a symptom of the subject on the basis of the examination deliverable.

In a case where the matching server 100 receives information (that is sometimes referred to as “estimation result information” below) regarding an estimation result of the symptom of the subject from the estimation server 400, the matching server 100 then transmits the estimation result information to the user terminal 500 and the management server 200. This allows the user to learn the estimation result of the symptom of the subject via the user terminal 500 and allows the management server 200 to suitably manage the estimation result information.

### (Management Server 200)

The management server 200 is a medical information processing apparatus that manages an examination deliverable, examination information corresponding to the examination deliverable, estimation result information regarding a symptom of a subject, and the like. The management of an examination deliverable and examination information is more specifically described. The management server 200 receives an examination deliverable, examination information, subject ID, and the like from the examination terminal 300 after an examination is carried out. The management server 200 then manages these pieces of information in association with each other. The subject ID is information that allows a subject to be identified.

The management of estimation result information regarding a symptom of a subject by the management server 200 is more specifically described. The management server 200 receives estimation result information regarding a symptom of a subject from the matching server 100 and manages the estimation result information and the subject ID in association with each other.

### (Examination Terminal 300)

The examination terminal 300 is a medical information processing apparatus that transmits an examination deliverable and the like to the management server 200. If described more specifically, the examination terminal 300 is an apparatus that is operated by a person in charge of medical examinations. The examination terminal 300 is operated by a person in charge of medical examinations (or automatically) to record an examination deliverable, examination information, and subject ID in association with each other and transmits these pieces of information to the management server 200 after an examination is finished. It is to be noted that it is also desirable to concurrently transmit consent information to the management server 200. The consent information indicates that the subject consents to the transmission of these pieces of information to the management server 200. It is to be noted that the examination terminal 300 is an apparatus that is operated by a user in a case where the user carries out an examination by himself or herself. In addition, the examination terminal 300 may be an examination device that is used for an examination. In addition, in a case where an examination deliverable and the like are provided to the management server 200 through communication with the user terminal 500 or in another method (e.g., by mail or the like), the medical information processing system does not have to include the examination terminal 300.

### (Estimation Server 400)

The estimation server 400 is a medical information processing apparatus that embodies an estimation system. The estimation server 400 is an apparatus that uses an examination deliverable to estimate a symptom of a subject. As described above, the estimation system is a system that estimates a symptom of a subject by using a machine learning algorithm which is an artificial intelligence algorithm. More specifically, the estimation server 400 inputs an examination deliverable provided from the matching server 100 to the machine learning algorithm, thereby obtaining the output of an estimation result of a symptom of a subject.

Here, the “estimation result of a symptom of a subject” includes information regarding the estimated injury and illness (including a disease and an injury and meaning that the normal physical function or shape is impaired), the severity of the injury and illness, a part having the injury and illness, a cause of the injury and illness, the probability (accuracy) of the estimation, or the like, but is not limited thereto.

The present embodiment assumes the presence of a plurality of estimation systems (not limited thereto). The estimation server 400 may thus embody a plurality of estimation systems (e.g., the estimation server 400 may have a program regarding the plurality of estimation systems) or the plurality of estimation servers 400 may be provided that corresponds to a plurality of respective estimation systems. As an example, the following describes, as an example, a case where the plurality of estimation servers 400 is provided that corresponds to a plurality of respective estimation systems (it is to be noted that FIG. 1 illustrates the one estimation server 400 alone for the sake of convenience).

### (User Terminal 500)

The user terminal 500 is an information processing apparatus (or a medical information processing apparatus) that is operated by a user. Here, the “user” is considered as at least one of a subject or a medical staff member (such as a doctor, a dentist, a pharmacist, a nurse, a midwife, a nutritionist, a physical therapist, or an occupational therapist, for example).

The user terminal 500 provides a user with a predetermined user interface by executing a predetermined program. Once the user makes a variety of inputs via the user interface, the user terminal 500 then transmits a signal (that is referred to as “matching request” below), selection information, and the like to the matching server 100 on the basis of the inputs. The matching request requests estimation system matching.

The generation of a matching request is more specifically described. A user selects an examination deliverable via a user interface. The examination deliverable is used to estimate a symptom. Afterward, the user terminal 500 generates a matching request including information (such as information that allows the examination deliverable to be identified, for example) indicating the selected examination deliverable and subject ID. It is to be noted that a matching request may include information other than information indicating a selected examination deliverable and subject ID. For example, the matching request may include setting information (such as what a user requests from an estimation system that is matched, a necessary item, or a limitation, for example) regarding an estimation system that is matched.

In addition, the user terminal 500 receives recommendation information (information regarding a matching result) and estimation result information regarding a symptom of a subject from the matching server 100 and provides these pieces of information to the user.

### (Network 600)

The network 600 is a network that couples the apparatuses described above through predetermined communication. It is to be noted that the network 600 does not necessarily have to couple all of the apparatuses, but may limit apparatuses that are able to communicate with each other. For example, the examination terminal 300 does not have to communicate with the user terminal 500, the matching server 100, or the like.

The communication schemes and the types of lines that are used for the network 600 are not particularly limited. For example, the network 600 may be achieved by a dedicated network such as IP-VPN (Internet Protocol-Virtual Private Network). In addition, the network 600 may be achieved by a public network such as the Internet, a telephone network, or a satellite communication network, and various LANs (Local Area Networks) including Ethernet (registered trademark), WAN (Wide Area Network), and the like. Further, the network 600 may be achieved by a wireless communication network of Wi-Fi (registered trademark), Bluetooth (registered trademark), or the like.

The system configuration example of the medical information processing system according to the present embodiment has been described above. It is to be noted that the system configuration described above with reference to FIG. 1 is merely an example. The system configuration of the medical information processing system according to the present embodiment is not limited to the example. For example, the functions of the respective apparatuses may be achieved by another apparatus. More specifically, all or a portion of the functions of the matching server 100 may be implemented in the management server 200. In contrast, all or a portion of the functions of the management server 200 may also be implemented in the matching server 100. It is possible to flexibly modify the system configuration of the medical information processing system according to the present embodiment in accordance with the specifications and operations.

### (1.2. Functional Component of Apparatus)

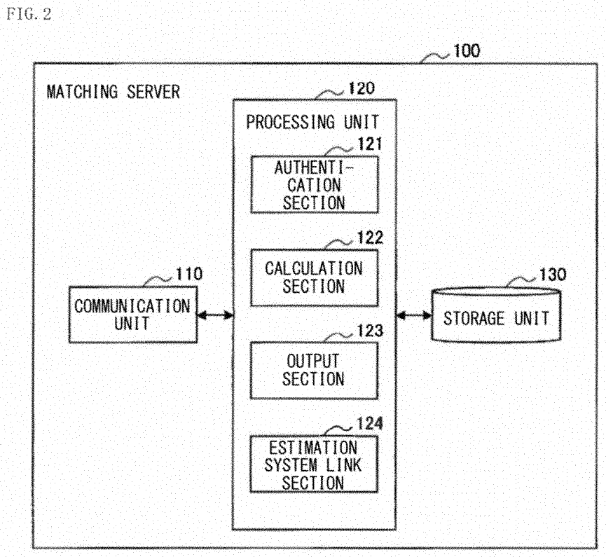

Next, with reference to FIGS. 2 to 5, functional component examples of the respective apparatuses included in the medical information processing system are described.

### (Functional Component Example of Matching Server 100)

First, with reference to FIG. 2, a functional component example of the matching server 100 is described. As illustrated in FIG. 2, the matching server 100 includes a communication unit 110, a processing unit 120, and a storage unit 130. In addition, the processing unit 120 includes an authentication section 121, a calculation section 122, an output section 123, and an estimation system link section 124.

The communication unit 110 is a functional component that communicates with an external apparatus. Communication with the user terminal 500 is described. The communication unit 110 receives a matching request, selection information, input information that is used for user authentication, and the like from the user terminal 500. The communication unit 110 transmits recommendation information, estimation result information, user authentication result information, and the like to the user terminal 500. In addition, communication with the estimation server 400 is described. The communication unit 110 transmits, for example, an examination deliverable and the like to the estimation server 400. The communication unit 110 receives estimation result information and the like from the estimation server 400. In addition, communication with the management server 200 is described. The communication unit 110 receives, for example, an examination deliverable, examination information, and the like from the management server 200 (i.e., the communication unit 110 also functions as an acquisition section that acquires examination information). The communication unit 110 transmits estimation result information and the like to the management server 200. It is to be noted that the information communicated by the communication unit 110 and a case where the communication unit 110 performs communication are not limited thereto.

The processing unit 120 is a functional component that comprehensively controls the overall process performed by the matching server 100. For example, the processing unit 120 is able to control a start and stop of each of the functional components. It is to be noted that the processing content of the processing unit 120 is not particularly limited. For example, the processing unit 120 may control a process (e.g., a process related to OS (Operating System), etc.) typically performed in a variety of servers, general purpose computers, PCs (Personal Computers), tablet PCs, or the like.

The authentication section 121 is a functional component that performs user authentication. More specifically, in a case where the user terminal 500 provides input information that is used for user authentication, the authentication section 121 performs a predetermined user authentication process by using the input information. It is to be noted that the type of user authentication is not particularly limited. For example, the authentication section 121 performs authentication in which identification information (such as ID, for example) of a user and a password are used, biometric authentication in which biometric information of a user is used, or the like. This allows the authentication section 121 to eliminate access from an unauthorized third person.

The calculation section 122 is a functional component that calculates the use priorities of a plurality of estimation systems on the basis of examination information and pieces of system information. More specifically, in a case where a matching request is provided from the user terminal 500, the calculation section 122 reads out a list of examination deliverables from the management server 200. The examination deliverables are associated with subject ID included in the matching request. The calculation section 122 then acquires an examination deliverable that is included in the matching request and selected by a user and examination information corresponding to the examination deliverable from the list of examination deliverables on the basis of information (such as information that allows the examination deliverable to be identified, for example) indicating the examination deliverable. Further, the calculation section 122 acquires pieces of system information regarding a plurality of estimation systems from the storage unit 130 (i.e., the calculation section 122 also functions as an acquisition section that acquires system information).

The calculation section 122 then calculates the use priorities of a plurality of estimation systems in accordance with a predetermined algorithm. For example, the calculation section 122 may reflect the degree to which an examination deliverable is adapted to the estimation system in the use priority by using “information regarding the details of the examination deliverable” included in examination information and “information regarding an examination deliverable necessary for the estimation system to estimate a symptom” included in system information. More specifically, the calculation section 122 may reflect the degree to which the type, data format, and the like of an examination deliverable are adapted to the estimation system in the use priority. In addition, the calculation section 122 may reflect the degree to which an examination and a device used for the examination are adapted to the estimation system in the use priority by using “information regarding the details of the examination” and “information regarding the device used for the examination” included in examination information and “information regarding learning data” and “information regarding a characteristic of the estimation system” included in system information. More specifically, the calculation section 122 may reflect the degree to which the type of examination, the product name of a device used for the examination, and the like are adapted to the estimation system in the use priority. It is to be noted that a method of calculating a use priority is not necessarily limited to the above as long as examination information and system information are used. In addition, the calculation section 122 may perform weighting or the like in accordance with the importance of each of various kinds of information included in examination information and system information. In addition, the calculation section 122 may calculate a use priority by using a machine learning algorithm.

The output section 123 is a functional component that outputs various kinds of information to an external apparatus. For example, in a case where the calculation section 122 calculates a use priority, the output section 123 outputs information (i.e., recommendation information) regarding at least one of a plurality of estimation systems to the user terminal 500 on the basis of the use priority. In a case where the recommendation information includes a plurality of estimation systems, the output section 123 then outputs each of the estimation systems to the user terminal 500 in a predetermined method. For example, the output section 123 may output pieces of information regarding a predetermined number of estimation systems in the decreasing order of use priorities to the user terminal 500. More specifically, the output section 123 may output the estimation system having the highest use priority alone or may output the top three estimation systems.

The output section 123 may then output an estimation system emphasized more with a higher use priority to facilitate a user to select the estimation system. Alternatively, the output section 123 may output various kinds of information (such as the cost and time necessary to estimate a symptom, the type of algorithm that is used for the estimation system, the administrator of the estimation system, or the user's use history of the estimation system, for example) regarding the estimation system together. It is to be noted that the output section 123 desirably outputs a predetermined icon for an estimation system having a use history in a case where a user's use history of an estimation system is outputted together. It is then desirable that the output section 123 make an output to display the details of a past use history once the user selects the icon.

In addition, in a case where an estimation system estimates a symptom of a subject, the output section 123 outputs estimation result information to the user terminal 500. In addition, in a case where the authentication section 121 performs user authentication, the output section 123 outputs a result of the user authentication to the user terminal 500. It is to be noted that the information outputted by the output section 123 and a case where the output section 123 makes an output are not limited thereto. In addition, an output method of the output section 123 may be flexibly changed in accordance with the specifications (functions or the like) of an output destination apparatus. For example, the output section 123 may change an output method in accordance with a mechanism (such as a display mechanism, an audio output mechanism, or a light emitting mechanism, for example) included in the output destination apparatus.

The estimation system link section 124 is a functional component that links the estimation system. More specifically, in a case where a user selects an estimation system (i.e., in a case where selection information is provided from the user terminal 500), the estimation system link section 124 reads out a list of examination deliverables associated with subject ID from the management server 200. The estimation system link section 124 then acquires an examination deliverable selected by the user from the list of examination deliverables and examination information corresponding to the examination deliverable. Further, the estimation system link section 124 performs a predetermined process (that is referred to as “personal information protection process” below) on the acquired examination deliverable. The personal information protection process makes personal information unidentifiable. The estimation system link section 124 then provides the examination deliverable subjected to the personal information protection process to an estimation system (i.e., estimation server 400 that embodies an estimation system) whose use is determined.

The personal information protection process is more specifically described. For example, in a case where the name or face image information of a subject is displayed on an examination deliverable, the estimation system link section 124 performs a blacking-out process or a data deletion process on the displayed portion. Even in a case where an examination deliverable includes personal information, this allows the estimation system link section 124 to provide the estimation server 400 with the examination deliverable on which the personal information is undecryptable. A method of achieving such a blacking-out process is described. For example, a classifier is generated by using learning data “with personal information” and learning data “without personal information” to perform learning. The learning data “with personal information” is data generated by superimposing the name, face image information, or the like of a subject on an examination deliverable. The learning data “without personal information” is data in which the name, face image information, or the like of a subject has not been superimposed. The estimation system link section 124 then inputs an examination deliverable to the classifier to determine the presence or absence of personal information of the subject such as the name or face image information and identify the position thereof and superimposes a black object at the position of the personal information to achieve a blacking-out process. It is to be noted that the contents of a personal information protection process are not limited to the above as long as it is possible to make personal information included in an examination deliverable undecryptable. In addition, a method of achieving a personal information protection process such as a blacking-out process is not limited to a method based on machine learning as described above.

In addition, the estimation system link section 124 provides the examination deliverable associated with the temporary ID to an estimation system (i.e., estimation server 400 that embodies an estimation system) whose use is determined. The temporary ID is temporarily used as masking ID. If described more specifically, the estimation system link section 124 generates ID as temporary ID. The ID is information that allows an examination deliverable to be identified and is different from subject ID. It is to be noted that a method of generating temporary ID is not particularly limited. For example, a publicly known temporary ID generation program or the like may be used. The estimation system link section 124 associates an examination deliverable and temporary ID in a predetermined method such as adding the temporary ID to the examination deliverable. The estimation system link section 124 provides the examination deliverable associated with the temporary ID to an estimation system (i.e., estimation server 400 that embodies an estimation system). It is to be noted that the estimation system link section 124 manages temporary ID and subject ID inside in association with each other.

After the estimation of a symptom of a subject is completed, the estimation system link section 124 then acquires estimation result information from an estimation system (i.e., estimation server 400 that embodies an estimation system). The estimation system link section 124 identifies the subject ID corresponding to the estimation result information on the basis of temporary ID associated with the estimation result information. The estimation system link section 124 associates the estimation result information and the subject ID. This makes it possible to identify the subject who is a target of the estimation result information.

The storage unit 130 is a functional component that stores various kinds of information. For example, the storage unit 130 stores system information that is used to calculate a use priority. In addition, the storage unit 130 stores information (such as a matching request, an examination deliverable, examination information, selection information, estimation result information, or input information that is used for user authentication, for example) provided from the user terminal 500, the management server 200, the estimation server 400, or the like or a processing result or the like (such as a use priority, for example) of each of the functional components of the matching server 100. In addition, the storage unit 130 stores a program, a parameter, or the like that is used by each of the functional components of the matching server 100. It is to be noted that the details of information stored in the storage unit 130 are not limited thereto.

The functional component example of the matching server 100 has been described above. It is to be noted that the functional component described above with reference to FIG. 2 is merely an example, but the functional component of the matching server 100 is not limited thereto. For example, the matching server 100 does not necessarily have to include all of the functional components illustrated in FIG. 2. In addition, the functional components of the matching server 100 are flexibly changeable in accordance with the specifications and operations.

### (Functional Component Example of Management Server 200)

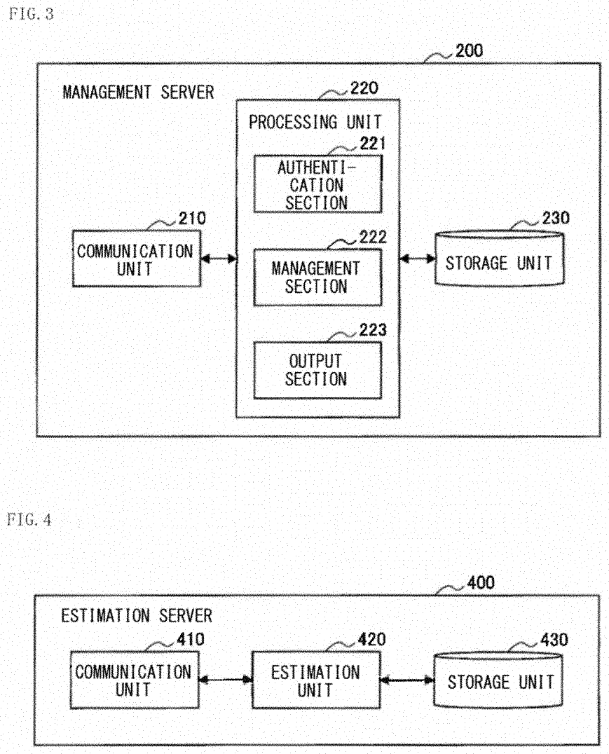

Next, with reference to FIG. 3, a functional component example of the management server 200 is described. As illustrated in FIG. 3, the management server 200 includes a communication unit 210, a processing unit 220, and a storage unit 230. In addition, the processing unit 220 includes an authentication section 221, a management section 222, and an output section 223.

The communication unit 210 is a functional component that communicates with an external apparatus. Communication with the examination terminal 300 is described. After an examination of a subject is finished, the communication unit 210 receives an examination deliverable and examination information corresponding to the examination deliverable from the examination terminal 300. Communication with the matching server 100 is described. The communication unit 210 receives subject ID and information indicating an examination deliverable from the matching server 100. The subject ID and the information indicating an examination deliverable are included in a matching request. The communication unit 210 transmits the examination deliverable and examination information corresponding to the examination deliverable to the matching server 100. In addition, after a symptom of a subject is estimated, the communication unit 210 receives estimation result information associated with the subject ID from the matching server 100. In addition, communication with the user terminal 500 is described. The communication unit 210 receives input information (e.g., in a case where the user terminal 500 directly accesses the management server 200 and the management server 200 performs user authentication) that is used for user authentication and information for requesting an examination deliverable and estimation result information from the user terminal 500. The communication unit 210 transmits user authentication result information and an examination deliverable and estimation result information requested by the user terminal 500 to the user terminal 500. It is to be noted that the information communicated by the communication unit 210 and a case where the communication unit 210 performs communication are not limited thereto.

The processing unit 220 is a functional component that comprehensively controls the overall process performed by the management server 200. For example, the processing unit 220 is able to control a start and stop of each of the functional components. It is to be noted that the processing content of the processing unit 220 is not particularly limited. For example, the processing unit 220 may control a process (such as a process regarding OS, for example) generally performed by each kind of server, general-purpose computer, PC, tablet PC, or the like.

The authentication section 221 is a functional component that performs user authentication. If described more specifically, the user terminal 500 sometimes directly accesses the management server 200 without going through the matching server 100. The authentication section 221 performs user authentication, for example, in this case. It is to be noted that the contents of user authentication performed by the authentication section 221 may be similar to those of user authentication performed by the authentication section 121 of the matching server 100 described above. Description is thus omitted.

The management section 222 is a functional component that manages an examination deliverable, examination information, and estimation result information. The management of an examination deliverable and examination information is more specifically described. In a case where the examination terminal 300 provides an examination deliverable, examination information corresponding to the examination deliverable, and subject ID, the management section 222 associates these pieces of information with each other and stores these pieces of information in the storage unit 230 in a predetermined format. In addition, the management section 222 may delete an examination deliverable or the like older than a predetermined period or replace an examination deliverable or the like of a similar examination carried out in the past with the latest examination deliverable or the like. In addition, in a case where an examination deliverable or the like is provided from the examination terminal 300, the management section 222 may calculate a fee on the basis of the contents of an examination and perform a process of charging the subject the fee. For example, in a case where credit card information or the like is registered as subject information, the management section 222 may perform a credit card payment process or the like on the basis of the information. In addition, in a case where a subject selects a link to an insurance company and an examination deliverable or the like is provided from the examination terminal 300, the management section 222 may notify an apparatus of the insurance company that the examination deliverable or the like is provided or provide the examination deliverable itself to an apparatus of the insurance company. This allows the subject to notify the insurance company of the frequency of examinations or an examination deliverable. The subject is thus able to receive a predetermined insurance service (such as a reduced insurance fee, for example).

The management of estimation result information is more specifically described. In a case where the matching server 100 provides estimation result information associated with subject ID, the management section 222 stores the information in the storage unit 230 in a predetermined format. In addition, the management section 222 may delete estimation result information or the like older than a predetermined period or replace estimation result information or the like generated by the same estimation system in the past with the latest estimation result information or the like. In addition, in a case where estimation result information is provided from the matching server 100, the management section 222 may calculate a fee on the basis of an estimation system or the like used to estimate a symptom and performs a process of charging a subject the fee. A specific example of the charging process is similar to the above and description is thus omitted.

The output section 223 is a functional component that outputs various kinds of information to an external apparatus. For example, the output section 223 outputs an examination deliverable, examination information, or estimation result information to the matching server 100 or the user terminal 500. In addition, in a case where the authentication section 221 performs user authentication, the output section 223 outputs a result of the user authentication to the user terminal 500. It is to be noted that the information outputted by the output section 223 and a case where the output section 223 makes an output are not limited thereto. In addition, an output method of the output section 223 may be flexibly changed in accordance with the specifications (functions or the like) of an output destination apparatus.

The storage unit 230 is a functional component that stores various kinds of information. For example, the storage unit 230 stores an examination deliverable, examination information, and estimation result information in a predetermined format. In addition, the storage unit 230 stores a program, a parameter, or the like that is used by each of the functional components of the management server 200. It is to be noted that the details of information stored in the storage unit 230 are not limited thereto.

The functional component example of the management server 200 has been described above. It is to be noted that the functional component described above with reference to FIG. 3 is merely an example, but the functional component of the management server 200 is not limited thereto. For example, the management server 200 does not necessarily have to include all of the functional components illustrated in FIG. 3. In addition, the functional components of the management server 200 are flexibly changeable in accordance with the specifications and operations.

### (Functional Component Example of Estimation Server 400)

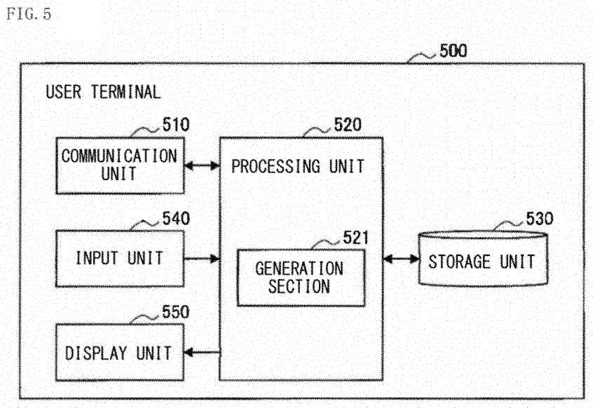

Next, with reference to FIG. 4, a functional component example of the estimation server 400 is described. As illustrated in FIG. 4, the estimation server 400 includes a communication unit 410, an estimation unit 420, and a storage unit 430.

The communication unit 410 is a functional component that communicates with an external apparatus. For example, the communication unit 410 receives an examination deliverable from the matching server 100. The examination deliverable has been subjected to a personal information protection process. After a process of estimating a symptom of the subject is performed on the basis of the examination deliverable, the communication unit 410 then transmits estimation result information to the matching server 100. It is to be noted that the information communicated by the communication unit 410 and a case where the communication unit 410 performs communication are not limited thereto.

The estimation unit 420 is a functional component that embodies an estimation system and estimates a symptom of a subject by using an examination deliverable provided from the matching server 100. More specifically, the estimation unit 420 inputs an examination deliverable to a machine learning algorithm, thereby obtaining the output of an estimation result of a symptom of a subject.

Here, the artificial intelligence algorithm is an algorithm that extrapolates on the basis of learning, statistics, or a predetermined rule. In addition, the machine learning algorithm is an algorithm that is one of population intelligence algorithms and extrapolates on the basis of a learning result. The machine learning algorithm is, for example, a classification model or a regression model that uses a neural network. It is to be noted that another technique such as SVM (Support Vector Machine) or a random forest may be used for the machine learning algorithm. In a case of the machine learning technique, for example, learning data in which a diagnosis result of a doctor and an examination deliverable are associated is inputted for learning to a predetermined calculation model that uses a neural network. A processing circuit including a processing model having a generated parameter may achieve the function of the machine learning algorithm. It is to be noted that a method of generating a machine learning algorithm used by the estimation unit 420 for a process is not limited to the above. It is to be noted that the function of the machine learning algorithm for classification or regression may be achieved by using another artificial intelligence algorithm.

In addition, as described above, the present embodiment assumes a case where the plurality of estimation servers 400 is provided that corresponds to a plurality of respective estimation systems (needless to say, not limited thereto). The estimation systems embodied by the estimation units 420 of the respective estimation servers 400 have algorithms (artificial intelligence algorithms) that are different from each other. This causes the respective estimation systems to have different characteristics.

The storage unit 430 is a functional component that stores various kinds of information. For example, the storage unit 430 stores an examination deliverable provided from the matching server 100, estimation result information outputted from the estimation unit 420, and the like. In addition, the storage unit 430 stores a program, a parameter, or the like that is used by each of the functional components of the estimation server 400. It is to be noted that the details of information stored in the storage unit 430 are not limited thereto.

The functional component example of the estimation server 400 has been described above. It is to be noted that the functional component described above with reference to FIG. 4 is merely an example, but the functional component of the estimation server 400 is not limited thereto. For example, the estimation server 400 does not necessarily have to include all of the functional components illustrated in FIG. 4. In addition, the functional components of the estimation server 400 are flexibly changeable in accordance with the specifications and operations.

### (Functional Component Example of User Terminal 500)

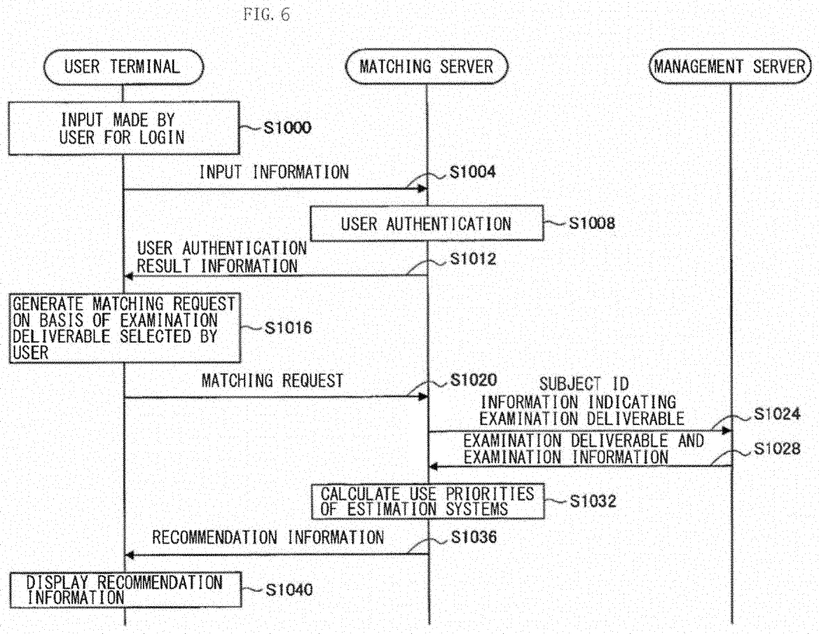

Next, with reference to FIG. 5, a functional component example of the user terminal 500 is described. As illustrated in FIG. 5, the user terminal 500 includes a communication unit 510, a processing unit 520, a storage unit 530, an input unit 540, and a display unit 550. In addition, the processing unit 520 includes a generation section 521.

The communication unit 510 is a functional component that communicates with an external apparatus. Communication with the matching server 100 is described. The communication unit 510 transmits a matching request, selection information, input information that is used for user authentication, and the like to the matching server 100. The communication unit 510 receives recommendation information, estimation result information, and user authentication result information from the matching server 100. In addition, communication with the management server 200 is described. The communication unit 510 transmits input information (e.g., in a case where the user terminal 500 directly accesses the management server 200 and the management server 200 performs user authentication) that is used for user authentication and information for requesting an examination deliverable, estimation result information, and the like to the management server 200. The communication unit 510 receives user authentication result information and an examination deliverable, estimation result information, and the like that are requested from the management server 200. It is to be noted that the information communicated by the communication unit 510 and a case where the communication unit 510 performs communication are not limited thereto.

The processing unit 520 is a functional component that comprehensively controls the overall process performed by the user terminal 500. For example, the processing unit 520 is able to control a start and stop of each of the functional components. It is to be noted that the processing content of the processing unit 520 is not particularly limited. For example, the processing unit 520 may control a process (such as a process regarding OS, for example) generally performed by each kind of server, general-purpose computer, PC, tablet PC, or the like.

The generation section 521 is a functional component that generates a matching request on the basis of an input made by a user. More specifically, the generation section 521 provides a user with a predetermined user interface by executing a predetermined program. A user selects an examination deliverable that is used to estimate a symptom via the user interface. Afterward, the generation section 521 generates a matching request including information (such as information that allows the examination deliverable to be identified, for example) indicating the selected examination deliverable and subject ID. It is to be noted that the generation section 521 may include, in a matching request, information other than information indicating a selected examination deliverable and subject ID. For example, the generation section 521 may include, in a matching request, setting information (such as what a user requests from an estimation system that is matched, a necessary item, or a limitation, for example) regarding an estimation system that is matched. The setting information is inputted by a user. It is to be noted that a specific example of a user interface provided by the generation section 521 to a user is described below.

The storage unit 530 is a functional component that stores various kinds of information. For example, the storage unit 530 stores information (such as recommendation information, estimation result information, user authentication result information, or an examination deliverable, for example) provided from the matching server 100, the management server 200, or the like or a processing result or the like (such as a matching request or selection information, for example) of each of the functional components of the user terminal 500. In addition, the storage unit 530 stores a program, a parameter, or the like that is used by each of the functional components of the user terminal 500. It is to be noted that the details of information stored in the storage unit 530 are not limited thereto.

The input unit 540 is a functional component that receives an input made by a user. For example, the input unit 540 includes an input device such as a mouse, a keyboard, a touch panel, a button, a switch, a microphone, or a camera. The use of these input devices allows a user to input desired information. It is to be noted that an input device included in the input unit 540 is not particularly limited.

The display unit 550 is a functional component that displays various kinds of information. More specifically, the display unit 550 includes a display device such as a display, a projection device such as a projector, or the like. The use of these devices makes it possible to provide a user with a processing result of the own apparatus or information or the like provided from the matching server 100, the management server 200, or the like. It is to be noted that a device included in the display unit 550 is not limited to the above.

The functional component example of the user terminal 500 has been described above. It is to be noted that the functional component described above with reference to FIG. 5 is merely an example, but the functional component of the user terminal 500 is not limited thereto. For example, the user terminal 500 does not necessarily have to include all of the functional components illustrated in FIG. 5. In addition, the functional components of the user terminal 500 are flexibly changeable in accordance with the specifications and operations.

### (1.3. Flow of Processes)

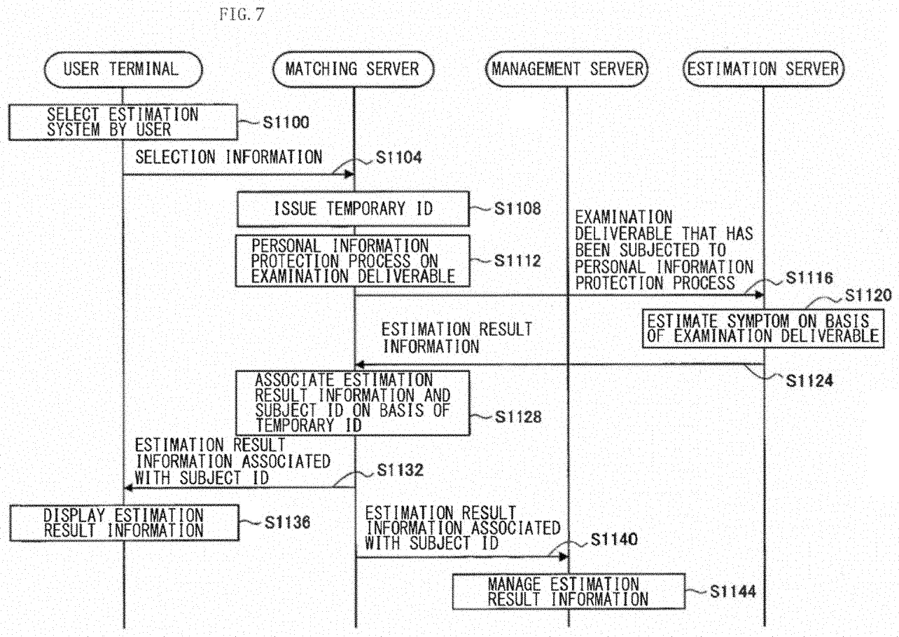

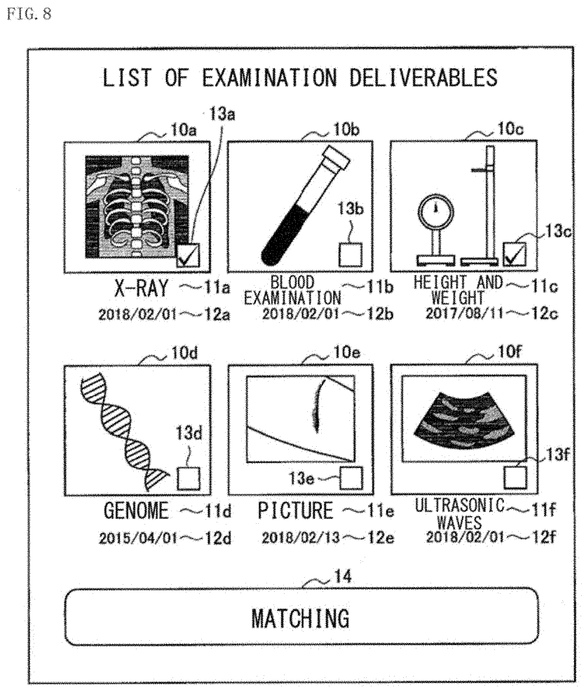

The above has described the functional component examples of the respective apparatuses included in the medical information processing system. Next, with reference to FIGS. 6 and 7, the flows of processes performed by the respective apparatuses included in the medical information processing system are described.

(Example of Flow of Processes of Matching Estimation System with Subject)

First, with reference to FIG. 6, an example of a flow of processes of matching an estimation system with a subject is described.

In step S1000, a user uses the input unit 540 of the user terminal 500 to make an input for logging in to the medical information processing system. For example, a user inputs identification information (such as ID, for example) of the user and a password or inputs biometric information for biometric authentication. It is to be noted that a function of the user terminal 500 may automate an input operation for login. In step S1004, the communication unit 510 transmits the input information inputted by the user to the matching server 100. For example, the communication unit 510 transmits identification information (such as ID, for example) of a user and hash pass information as input information. The hash pass information is obtained by hashing a password.

In step S1008, the authentication section 121 of the matching server 100 uses the input information to perform a predetermined user authentication process. For example, the authentication section 121 performs user authentication on the basis of whether or not the hash pass information provided as the input information matches hash pass information registered in advance. In step S1012, the output section 123 outputs user authentication result information to the user terminal 500 via the communication unit 110.

In step S1016, the generation section 521 of the user terminal 500 generates a matching request on the basis of an examination deliverable selected by the user. In step S1020, the communication unit 510 transmits the matching request to the matching server 100.

In step S1024, the calculation section 122 of the matching server 100 transmits information (such as information that allows the examination deliverable to be identified, for example) indicating the examination deliverable selected by the user and subject ID to the management server 200. The information is included in the matching request. In step S1028, the output section 223 of the management server 200 acquires the examination deliverable and examination information on the basis of the information indicating the examination deliverable and the subject ID. The output section 223 of the management server 200 outputs these pieces of information to the matching server 100.

In step S1032, the calculation section 122 of the matching server 100 calculates the use priorities of estimation systems on the basis of the pieces of system information stored in the storage unit 130 and the examination information provided from the management server 200. In step S1036, the output section 123 outputs information (i.e., recommendation information) regarding at least one of a plurality of estimation systems to the user terminal 500 on the basis of the use priority. In step S1040, the display unit 550 of the user terminal 500 displays recommendation information, thereby finishing the series of matching processes.

(Example of Flow of Processes regarding Estimation of Symptom)

Next, with reference to FIG. 7, an example of a flow of processes regarding the estimation of a symptom is described. FIG. 7 illustrates an example of a flow of processes performed after step S1040 (display of recommendation information) in FIG. 6.

In step S1100, a user uses the input unit 540 of the user terminal 500 to select at least one estimation system from the estimation systems included in recommendation information. In step S1104, the communication unit 510 transmits selection information indicating the selected estimation system to the matching server 100.

In step S1108, the estimation system link section 124 of the matching server 100 issues temporary ID that is temporarily used as masking ID. In step S1112, the estimation system link section 124 performs a personal information protection process on an examination deliverable. For example, in a case where the name or face image information of a subject is displayed on an examination deliverable, the estimation system link section 124 performs a blacking-out process or a data deletion process on the displayed portion. In step S1116, the estimation system link section 124 provides the estimation server 400 with the examination deliverable that has been subjected to a personal information protection process.

In step S1120, the estimation unit 420 of the estimation server 400 estimates a symptom of the subject on the basis of the examination deliverable. For example, the estimation unit 420 inputs an examination deliverable to a machine learning algorithm that is an artificial intelligence algorithm, thereby obtaining the output of estimation result information of a symptom of a subject. In step S1124, the communication unit 410 transmits the estimation result information to the matching server 100.

In step S1128, the estimation system link section 124 of the matching server 100 identifies the subject ID corresponding to the estimation result information on the basis of the temporary ID associated with the estimation result information. The estimation system link section 124 associates the estimation result information and the subject ID. In step S1132, the output section 123 outputs the estimation result information associated with the subject ID to the user terminal 500. In step S1136, the display unit 550 of the user terminal 500 displays the estimation result information.

In step S1140, the output section 123 of the matching server 100 outputs the estimation result information associated with the subject ID to the management server 200. In step S1144, the management section 222 of the management server 200 manages the estimation result information, thereby finishing the series of processes regarding the estimation of a symptom.

### (1.4. User Interface)

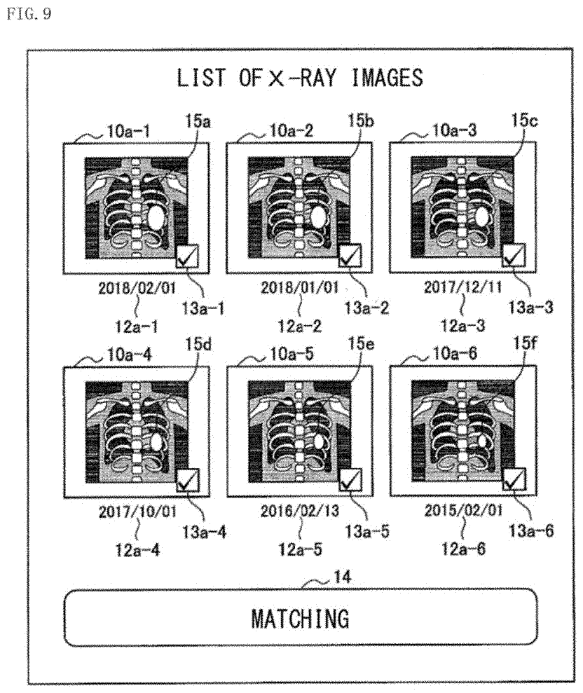

The above has described the flows of processes performed by the respective apparatuses included in the medical information processing system. Next, an example of a user interface provided by the generation section 521 of the user terminal 500 to a user is described. More specifically, with reference to FIGS. 8 to 10, an example of a user interface is described that is used to generate a matching request.

FIG. 8 is an example of a user interface that is used for a user to select an examination deliverable in generating a matching request. As illustrated in FIG. 8, there may be provided displays 10 (display 10a to display 10f) indicating the types of examination deliverables, names 11 (name 11a to name 11f) of the types of examination deliverables, latest examination dates 12 (examination date 12a to examination date 12f), check boxes 13 (check box 13a to check box 13f), and a matching button 14 as a user interface.

A user is able to designate examination deliverables that are used to estimate symptoms by inputting checks to the check boxes 13 (check box 13a to check box 13f). The user then pushes down the matching button 14 after designating at least one examination deliverable. This allows the user to generate a matching request and provide the matching request to the matching server 100.

Providing the displays 10 (display 10a to display 10f) indicating the types of examination deliverables allows a user to intuitively select an examination deliverable. In addition, providing the latest examination dates 12 (examination date 12a to examination date 12f) allows a user to easily determine from the perspective of examination dates whether or not the respective examination deliverables are suitable to estimate the symptoms.

It is to be noted that the generation section 521 may provide a predetermined warning to a user on the basis of the type of examination deliverable or an examination date in a case where the user designates an examination deliverable. For example, in a case where a plurality of types of examination deliverables is designated and the examination dates for the respective examination deliverables have predetermined intervals or more, the generation section 521 may determine that the estimation accuracy may possibly decrease and may provide a predetermined warning to a user. More specifically, in a case where X-ray image information and height/weight information are designated, the examination date of an X-ray examination is 2018/02/01, and the examination date (measurement date) for height/weight is 2017/08/11, the generation section 521 may provide a predetermined warning to a user on the basis that these examination dates have three months or more in between. This allows the user to carry out an examination again, select a different examination deliverable, and estimate a symptom or give up estimating a symptom by recognizing the possibility of a decrease in the estimation accuracy. Here, the contents of some (such as a genome test, for example) of examination deliverables are not changed at all (or not changed considerably) by the examination dates. Accordingly, it is desirable that the generation section 521 set the “predetermined intervals” described above in accordance with the types of examination deliverables. The “predetermined intervals” are used to determine whether or not it is necessary to issue a warning.

In addition, a symptom is sometimes estimated on the basis of a change in an examination deliverable over time. The generation section 521 may thus provide a user interface that allows a plurality of examination deliverables to be selected which is different in examination date. For example, as illustrated in FIG. 9, a user may use the user interface to select the pieces of X-ray image information from 2018/02/01 to 2015/02/01 and this may cause a symptom to be estimated on the basis of a change in the pieces of X-ray image information (e.g., a tumor 15a to a tumor 15f appearing in the respective pieces of X-ray image information in FIG. 9).

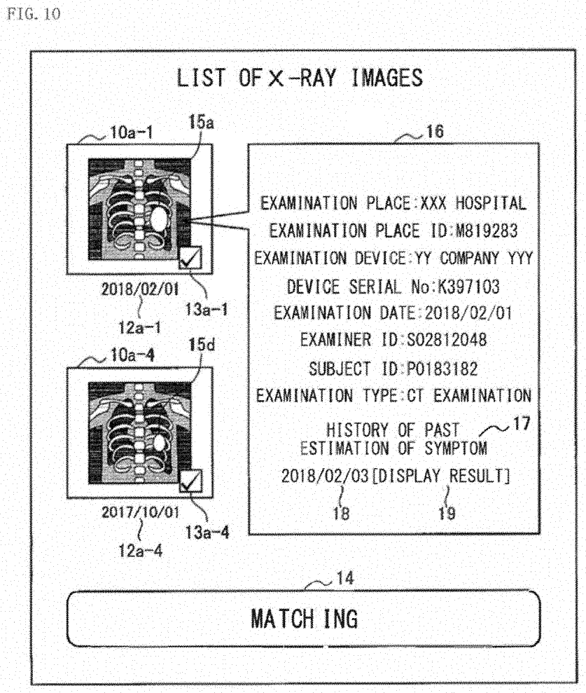

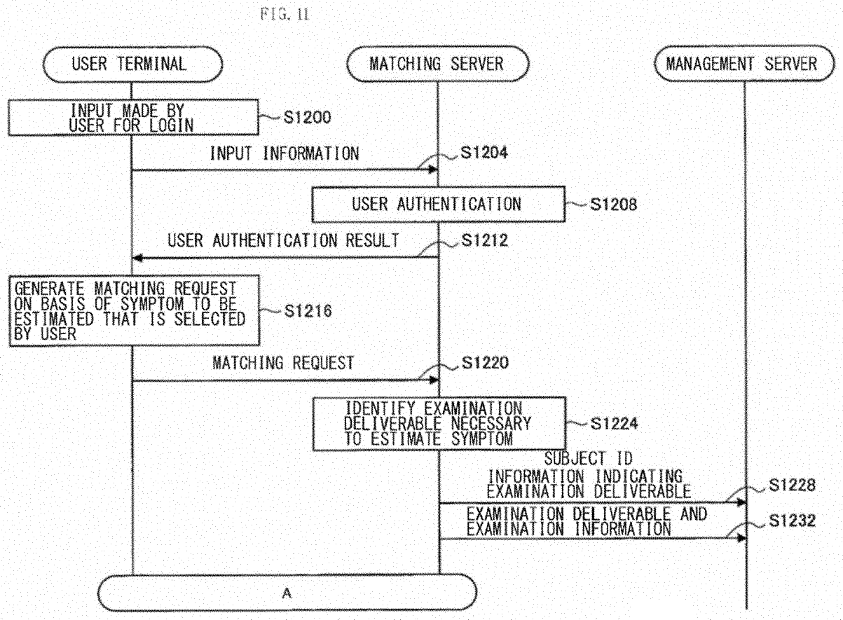

In addition, the generation section 521 may provide a user interface that makes it possible to confirm various kinds of information regarding examination deliverables (or various kinds of information regarding examinations). For example, in a case where a user makes a predetermined input such as holding the display 10 indicating the type of examination deliverable, the generation section 521 may provide a user interface that displays an examination place, information (illustrated as “examination place ID” in the diagram) that allows the examination place to be identified, an examination device, information (illustrated as “device serial No” in the diagram) that allows the examination device to be identified, an examination date, information (illustrated as “examiner ID” in the diagram) that allows an examiner to be identified, subject ID, or an examination type as illustrated in FIG. 10.

In addition, the generation section 521 may provide a user interface that makes it possible to confirm information 17 regarding a history of the estimation of a symptom performed in the past on the basis of the same examination deliverable as illustrated in FIG. 10. For example, the generation section 521 may provide a date 18 at which a symptom is estimated on the basis of the same examination deliverable and a link 19 for displaying estimation result information. As illustrated in FIG. 10, providing various kinds of information regarding examination deliverables (or various kinds of information regarding examinations), information regarding a history of the estimation of a symptom performed in the past, or the like allows a user to more suitably select an examination deliverable that is used to estimate a symptom.

### 2. Modification Example

The above has described the embodiment of the present disclosure. Next, a modification example of the present disclosure is described.

In the embodiment described above, a matching request is generated on the basis of an examination deliverable selected by a user. In contrast, in the modification example of the present disclosure, a matching request is generated on the basis of a symptom to be estimated that is selected by a user. If described more specifically, a user sometimes fails to recognize which of a plurality of examination deliverables is suitable to a process of estimating a symptom. In addition, a symptom to be estimated is sometimes determined in advance. Examples of such a case include a case where a user has a subjective symptom for a specific symptom. For example, it is sometimes determined in advance that a user wishes to estimate a “symptom caused by a headache” in a case where the user has a headache. Accordingly, in the modification example of the present disclosure, a user selects a symptom to be estimated to generate a matching request. The matching server 100 confirms the presence or absence of an examination deliverable necessary to estimate the symptom on the basis of the matching request. The matching server 100 performs a matching process in the presence of the necessary examination deliverable. The matching server 100 performs a predetermined process (such as notifying the user thereof, proposing an examination, or arranging an examination, for example) in the absence of the necessary examination deliverable.

Here, the “symptom to be estimated” that is selected by a user may be, for example, an injury and illness (including a disease and an injury). Alternatively, it is sufficient if the “symptom to be estimated” that is selected by a user indicates a symptom in some method such as a “symptom caused by a headache”.

The system configuration example according to the modification example may be similar to the system configuration example according to the embodiment described above with reference to FIG. 1 (they do not necessarily have be the same). Description is thus omitted.

In addition, functional component examples of the respective apparatuses are described. The generation section 521 of the user terminal 500 provides a user interface that allows a user to select a symptom to be estimated. For example, the generation section 521 may provide an application (that is sometimes referred to as “inquiry application” below), radio buttons, a text box, and the like. The inquiry application allows a user to narrow down symptoms to be estimated by answering one or more questions (i.e., making inquiries and responses). The radio buttons make it possible to select one or more symptoms to be estimated. It is possible to simply input a symptom to be estimated to the text box. It is to be noted that the generation section 521 may achieve the inquiry application by using a machine learning algorithm. A user is able to suitably select a symptom to be estimated via these user interfaces and generate a matching request including information indicating the symptom.

In a case where a symptom to be estimated is determined on the basis of an input made by a user (i.e., in a case where a matching request is generated), the calculation section 122 of the matching server 100 then confirms whether an examination deliverable that is used to estimate the symptom suffices. In a case where the examination deliverable suffices, the matching server 100 calculates a use priority. If described more specifically, the calculation section 122 first identifies an examination deliverable necessary to estimate a symptom to be estimated on the basis of information that is included in the matching request provided from the user terminal 500 and indicates the symptom. For example, the storage unit 130 of the matching server 100 stores a list of examination deliverables necessary to estimate a variety of symptoms in advance. The calculation section 122 acquires the information to identify an examination deliverable necessary to estimate a symptom designated by a matching request. The calculation section 122 then reads out a list of examination deliverables associated with subject ID included in the matching request from the management server 200. Afterward, the calculation section 122 acquires an examination deliverable necessary to estimate a symptom and examination information corresponding to the examination deliverable from the list of examination deliverables. In a case where all of the examination deliverables necessary to estimate a symptom are prepared, the calculation section 122 then calculates the use priorities of estimation systems on the basis of pieces of examination information and pieces of system information. In contrast, in a case where the examination deliverables necessary to estimate a symptom are not prepared, a predetermined process (such as notifying the user thereof, proposing an examination, or arranging an examination, for example) is performed. It is to be noted that the other functional components may be similar to those of the embodiment described above (they do not necessarily have to be the same). Description is thus omitted.

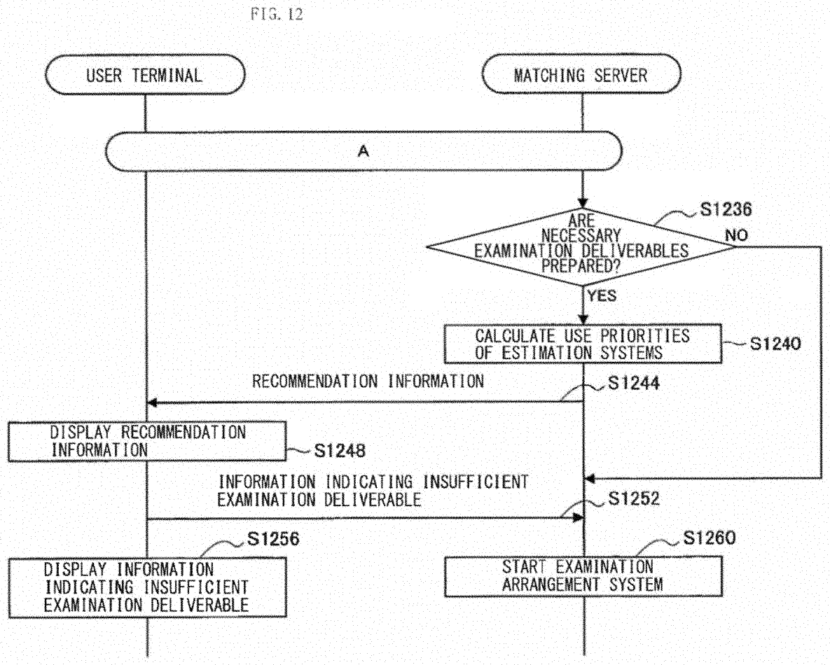

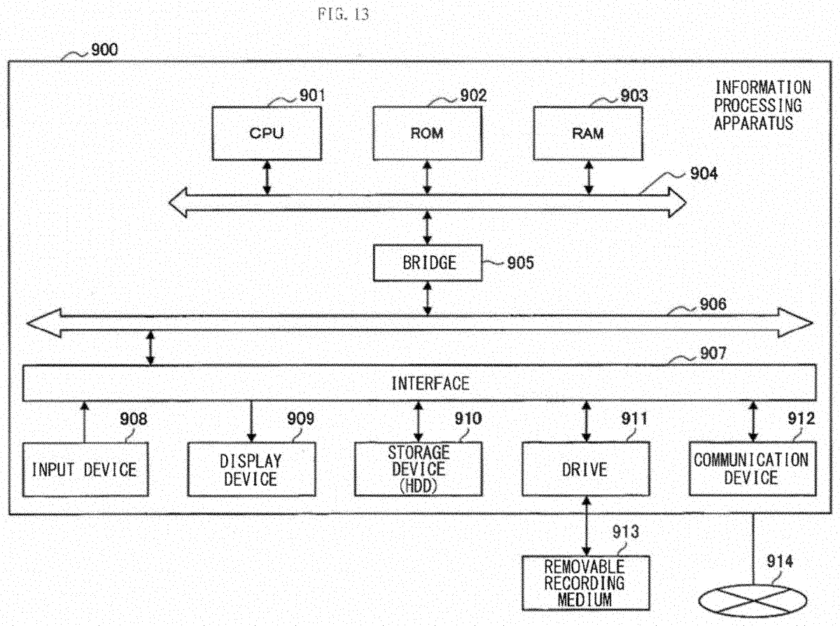

Next, with reference to FIGS. 11 and 12, an example of a flow of processes of matching an estimation system according to the modification example with a subject is described.

In step S1200, a user uses the input unit 540 of the user terminal 500 to make an input for logging in to the medical information processing system. In step S1204, the communication unit 510 transmits the input information inputted by the user to the matching server 100. In step S1208, the authentication section 121 of the matching server 100 uses the input information to perform a predetermined user authentication process. In step S1212, the output section 123 outputs user authentication result information to the user terminal 500 via the communication unit 110.

In step S1216, the generation section 521 of the user terminal 500 generates a matching request on the basis of a symptom to be estimated that is selected by the user. In step S1220, the communication unit 510 transmits the matching request to the matching server 100.

In step S1224, the calculation section 122 of the matching server 100 identifies an examination deliverable necessary to estimate a symptom designated by the matching request. In step S1228, the calculation section 122 of the matching server 100 transmits information (such as information that allows the examination deliverable to be identified, for example) indicating the identified examination deliverable and subject ID to the management server 200. In step S1232, the output section 223 of the management server 200 acquires the examination deliverable and examination information on the basis of the information indicating the examination deliverable and the subject ID. The output section 223 of the management server 200 outputs these pieces of information to the matching server 100.

In step S1236, the calculation section 122 of the matching server 100 confirms whether or not all of the examination deliverables necessary to estimate the symptom are prepared. In a case where all of the necessary examination deliverables are prepared (step S1236/Yes), the calculation section 122 calculates the use priorities of estimation systems in step S1240 on the basis of the pieces of system information stored in the storage unit 130 and the pieces of examination information provided from the management server 200. In step S1244, the output section 123 outputs information (i.e., recommendation information) regarding at least one of a plurality of estimation systems to the user terminal 500 on the basis of the use priority. In step S1248, the display unit 550 of the user terminal 500 displays the recommendation information.

In a case where the necessary examination deliverables are not prepared in step S1236 (step S1236/No), the matching server 100 performs a predetermined process. For example, the output section 123 outputs information indicating an insufficient examination deliverable to the user terminal 500 in step S1252. The display unit 550 of the user terminal 500 displays the information in step S1256. This allows a user to recognize that examination deliverables necessary to estimate a symptom are insufficient. In addition, in step S1260, the processing unit 120 of the matching server 100 may start a system (illustrated as “examination arrangement system” in the diagram) that arranges an examination (or proposes an examination, for example) for an insufficient examination deliverable) and arrange an examination (or propose an examination, for example). The series of matching processes are finished as described above.

### 3. Hardware Configuration

The above has described the modification example according to the present disclosure. Next, with reference to FIG. 13, a hardware configuration example of an information processing apparatus 900 is described. The information processing apparatus 900 embodies the matching server 100, the management server 200, an examination terminal 300, the estimation server 400, or the user terminal 500.

FIG. 13 is a diagram illustrating a hardware configuration of the information processing apparatus 900. The information processing apparatus 900 includes CPU (Central Processing Unit) 901, ROM (Read Only Memory) 902, RAM (Random Access Memory) 903, a host bus 904, a bridge 905, an external bus 906, an interface 907, an input device 908, an output device 909, a storage device (HDD) 910, a drive 911, and a communication device 912.

The CPU 901 functions as an arithmetic processing device and a control device. The CPU 901 controls the overall operation in the information processing apparatus 900 in accordance with a variety of programs. In addition, the CPU 901 may be a microprocessor. The ROM 902 stores a program, an arithmetic parameter, and the like to be used by the CPU 901. The RAM 903 temporarily stores a program used in execution of the CPU 901, a parameter appropriately changed in the execution, and the like. These components are coupled to each other by the host bus 904 including a CPU bus and the like. The CPU 901, the ROM 902, and the RAM 903 cooperate with each other to achieve the respective functions of the processing unit 120 of the matching server 100, the processing unit 220 of the management server 200, a processing unit (not illustrated) of the examination terminal 300, the estimation unit 420 of the estimation server 400, or the processing unit 520 of the user terminal 500.

The host bus 904 is coupled to the external bus 906 such as a PCI (Peripheral Component Interconnect/Interface) bus via the bridge 905. It is to be noted that the host bus 904, the bridge 905, and the external bus 906 does not have to be included separately, but the functions thereof may be implemented in one bus.

The input device 908 includes input means such as a mouse, a keyboard, a touch panel, a button, a microphone, a switch, and a lever for a user to input information, an input control circuit that generates an input signal on the basis of an input made by the user and outputs the generated input signal to the CPU 901, and the like. The user is able to input various kinds of information to the respective devices and instruct the respective devices about processing operations by operating the input device 908. The input device 908 achieves a function of the input unit 540 of the user terminal 500.

The output device 909 includes, for example, a display device such as a CRT (Cathode Ray Tube) display device, a liquid crystal display (LCD) device, an OLED (Organic Light Emitting Diode) device, and a lamp. The output device 909 further includes an audio output device such as a speaker and headphones. The display device displays various kinds of data as text or images. The audio output device converts various kinds of data into sounds and outputs the sounds. The output device 909 achieves a function of the display unit 550 of the user terminal 500.

The storage device 910 is a device for storing data. The storage device 910 may include a storage medium, a recording device that records data in the storage medium, a reading device that reads out data from the storage medium, a deleting device that deletes data recorded in the storage medium, and the like. The storage device 910 includes, for example, HDD (Hard Disk Drive). This storage device 910 drives a hard disk and stores a program to be executed by the CPU 901 and various kinds of data. The storage device 910 achieves the respective functions of the storage unit 130 of the matching server 100, the storage unit 230 of the management server 200, a storage unit (not illustrated) of the examination terminal 300, the storage unit 430 of the estimation server 400, or the storage unit 530 of the user terminal 500.

The drive 911 is a reader/writer for a storage medium. The drive 911 is built in or externally attached to the information processing apparatus 900. The drive 911 reads out information recorded on a mounted removable storage medium 913 and outputs the read information to the RAM 903. The removable storage medium 913 includes a magnetic disk, an optical disc, a magneto-optical disk, a semiconductor memory, and the like. In addition, the drive 911 is also able to write information to the removable storage medium 913.

The communication device 912 is, for example, a communication interface including a communication device and the like for coupling to a communication network 914. The communication device 912 achieves the respective functions of the communication unit 110 of the matching server 100, the communication unit 210 of the management server 200, a communication unit (not illustrated) of the examination terminal 300, the communication unit 410 of the estimation server 400, or the communication unit 510 of the user terminal 500.

### 4. Conclusion

As described above, the matching server 100 according to the present disclosure is able to acquire examination information regarding an examination and pieces of system information regarding a plurality of respective estimation systems. The matching server 100 according to the present disclosure is able to calculate the use priorities of the plurality of estimation systems on the basis of the examination information and the pieces of system information. The matching server 100 according to the present disclosure is able to output recommendation information regarding at least one of the plurality of estimation systems to a user on the basis of the use priorities. This allows the user to select a more suitable estimation system. In other words, a user is able to use an estimation system having higher accuracy among a plurality of estimation systems. This makes it possible to suppress the personnel expenses for a diagnostician, for example, by preventing a subject from going to a medical institution or gaining a second opinion from the estimation system, for example, in a case where the subject develops a minor symptom. It is thus possible to more suitably suppress increasing medical expenses.

A preferred embodiment(s) of the present disclosure has/have been described above in detail with reference to the accompanying drawings, but the technical scope of the present disclosure is not limited to such an embodiment(s). A person skilled in the art may find various alterations and modifications within the scope of the appended claims, and it should be understood that they will naturally come under the technical scope of the present disclosure.

In addition, the effects described herein are merely illustrative and exemplary, but not limitative. In other words, the technology according to the present disclosure may exert other effects that are apparent to those skilled in the art from the description herein in addition to the above-described effects or in place of the above-described effects.

It is to be noted that the following configurations also fall within the technical scope of the present disclosure.

(1)

A medical information processing system including:

an acquisition section that acquires examination information and system information, the examination information pertaining to an examination from which an examination deliverable regarding medical care is generated, the system information pertaining to each of a plurality of estimation systems, the plurality of estimation systems each estimating a symptom of a subject on the basis of the examination deliverable; and

a calculation section that calculates a use priority of each of the plurality of estimation systems on the basis of the examination information and the system information.

(2)

The medical information processing system according to (1), in which the estimation system estimates the symptom of the subject by using a machine learning algorithm.

(3)

The medical information processing system according to (2), in which the system information includes at least one of information regarding learning data, information regarding a symptom estimatable by the estimation system, information regarding a characteristic of the estimation system, or information regarding the examination deliverable necessary for the estimation system to estimate a symptom, the learning data being used for the machine learning algorithm.

(4)

The medical information processing system according to any one of (1) to (3), in which the examination information includes at least one of information regarding a detail of the examination, information regarding a device used for the examination, or information regarding a detail of the examination deliverable.

(5)

The medical information processing system according to any one of (1) to (4), further including an output section that outputs information regarding at least one of the plurality of estimation systems to a user on the basis of the use priorities.

(6)

The medical information processing system according to (5), in which the output section outputs pieces of information regarding a predetermined number of the estimation systems in a decreasing order of the use priorities to the user.

(7)

The medical information processing system according to (5) or (6), in which the user includes at least one of the subject or a medical staff member.

(8)

The medical information processing system according to any one of (5) to (7), in which the acquisition section also acquires the examination deliverable, and the medical information processing system further includes an estimation system link section that provides the examination deliverable to an estimation system whose use is determined on the basis of an input made by the user.

(9)

The medical information processing system according to (8), in which the estimation system link section provides the examination deliverable associated with temporary ID to the estimation system whose use is determined, the temporary ID being temporarily used as masking ID.

(10)

The medical information processing system according to (8) or (9), in which the estimation system link section provides the examination deliverable subjected to a personal information protection process to the estimation system whose use is determined, the personal information protection process making personal information unidentifiable.

(11)

The medical information processing system according to any one of (8) to (10), in which

the estimation system link section acquires information regarding an estimation result of the symptom of the subject from the estimation system whose use is determined, and

the output section outputs the information regarding the estimation result of the symptom of the subject to the user.

(12)

The medical information processing system according to any one of (1) to (11), in which the calculation section confirms whether the examination deliverable suffices in a case where a symptom to be estimated is determined on the basis of an input made by a user and the calculation section calculates the use priority in a case where the examination deliverable suffices, the examination deliverable being used to estimate the symptom.

(13)

A medical information processing apparatus including:

an acquisition section that acquires examination information and system information, the examination information including information pertaining to an examination from which an examination deliverable of a subject regarding medical care is generated, the system information including information pertaining to each of a plurality of estimation systems, the plurality of estimation systems each estimating a symptom of the subject on the basis of the examination deliverable; and

a calculation section that calculates a use priority of each of the plurality of estimation systems on the basis of the examination information and the system information.

(14)

A medical information processing method that is executed by a computer, the medical information processing method including:

acquiring examination information and system information, the examination information including information pertaining to an examination from which an examination deliverable of a subject regarding medical care is generated, the system information including information pertaining to each of a plurality of estimation systems, the plurality of estimation systems each estimating a symptom of the subject on the basis of the examination deliverable; and

calculating a use priority of each of the plurality of estimation systems on the basis of the examination information and the system information.

### REFERENCE SIGNS LIST

100 matching server
110 communication unit
120 processing unit
121 authentication section
122 calculation section
123 output section
124 estimation system link section
130 storage unit
200 management server
210 communication unit
220 processing unit
221 authentication section
222 management section
223 output section
230 storage unit
300 examination terminal
400 estimation server
410 communication unit
420 estimation unit
430 storage unit
500 user terminal
510 communication unit
520 processing unit
521 generation section
530 storage unit
540 input unit
550 display unit
600 network

1. A medical information processing system comprising:
an acquisition section that acquires examination information and system information, the examination information pertaining to an examination from which an examination deliverable regarding medical care is generated, the system information pertaining to each of a plurality of estimation systems, the plurality of estimation systems each estimating a symptom of a subject on a basis of the examination deliverable; and
a calculation section that calculates a use priority of each of the plurality of estimation systems on a basis of the examination information and the system information.

2. The medical information processing system according to claim 1, wherein the estimation system estimates the symptom of the subject by using a machine learning algorithm.

3. The medical information processing system according to claim 2, wherein the system information includes at least one of information regarding learning data, information regarding a symptom estimatable by the estimation system, information regarding a characteristic of the estimation system, or information regarding the examination deliverable necessary for the estimation system to estimate a symptom, the learning data being used for the machine learning algorithm.

4. The medical information processing system according to claim 1, wherein the examination information includes at least one of information regarding a detail of the examination, information regarding a device used for the examination, or information regarding a detail of the examination deliverable.

5. The medical information processing system according to claim 1, further comprising an output section that outputs information regarding at least one of the plurality of estimation systems to a user on a basis of the use priorities.

6. The medical information processing system according to claim 5, wherein the output section outputs pieces of information regarding a predetermined number of the estimation systems in a decreasing order of the use priorities to the user.

7. The medical information processing system according to claim 5, wherein the user includes at least one of the subject or a medical staff member.

8. The medical information processing system according to claim 5, wherein
the acquisition section also acquires the examination deliverable, and
the medical information processing system further includes an estimation system link section that provides the examination deliverable to an estimation system whose use is determined on a basis of an input made by the user.

9. The medical information processing system according to claim 8, wherein the estimation system link section provides the examination deliverable associated with temporary ID to the estimation system whose use is determined, the temporary ID being temporarily used as masking ID.

10. The medical information processing system according to claim 8, wherein the estimation system link section provides the examination deliverable subjected to a personal information protection process to the estimation system whose use is determined, the personal information protection process making personal information unidentifiable.

11. The medical information processing system according to claim 8, wherein
the estimation system link section acquires information regarding an estimation result of the symptom of the subject from the estimation system whose use is determined, and
the output section outputs the information regarding the estimation result of the symptom of the subject to the user.

12. The medical information processing system according to claim 1, wherein the calculation section confirms whether the examination deliverable suffices in a case where a symptom to be estimated is determined on a basis of an input made by a user and the calculation section calculates the use priority in a case where the examination deliverable suffices, the examination deliverable being used to estimate the symptom.

13. A medical information processing apparatus comprising:
an acquisition section that acquires examination information and system information, the examination information including information pertaining to an examination from which an examination deliverable of a subject regarding medical care is generated, the system information including information pertaining to each of a plurality of estimation systems, the plurality of estimation systems each estimating a symptom of the subject on a basis of the examination deliverable; and
a calculation section that calculates a use priority of each of the plurality of estimation systems on a basis of the examination information and the system information.

14. A medical information processing method that is executed by a computer, the medical information processing method comprising:
acquiring examination information and system information, the examination information including information pertaining to an examination from which an examination deliverable of a subject regarding medical care is generated, the system information including information pertaining to each of a plurality of estimation systems, the plurality of estimation systems each estimating a symptom of the subject on a basis of the examination deliverable; and
calculating a use priority of each of the plurality of estimation systems on a basis of the examination information and the system information.
